(19) United States
(12) Patent Application Publication
(10) Pub. No.: US 2008/0095867 A1
(43) Pub. Date: Apr. 24, 2008

(54) HYDROXYCITRIC ACID COMPOSITIONS FROM GARCINIA CAMBOGIA AND HIBISCUS SP., METHODS OF MAKING, AND THERAPEUTIC USES OF SAME

(75) Inventor: Scott Alexander Moffett, Chatsworth, CA (US)

Correspondence Address: GREENBERG TRAURIG LLP (LA), 2450 COLORADO AVENUE, SUITE 400E, INTELLECTUAL PROPERTY DEPARTMENT, SANTA MONICA, CA, 90404, US

(73) Assignee: RENAISSANCE HERBS, INC., Chatsworth, CA (US)

(21) Appl. No.: 11/875,680

(22) Filed: Oct. 19, 2007

Related U.S. Application Data
(60) Provisional application No. 60/862,171, filed on Oct. 19, 2006.

(51) Int. Cl. A61K 36/185; A61K 36/45; A61K 36/82; A61K 36/87; A61P 3/00; A61P 43/00

(52) U.S. Cl. 424/729; 424/725; 424/730; 424/766

(57) ABSTRACT

Herbal compositions that comprise one or more of extracts of hydroxycitric acid, (−) HCA and (+) HCA, from Garcinia cambogia and Hibiscus sabdariffa, and anthocyanins from Hibiscus sabdariffa; all constituent extracts at independently controlled concentrations which optionally include second therapeutic agents and pharmaceutically acceptable additives. Processes for the preparation of these herbal compositions, such processes including steps of extracting hydroxycitric acids and anthocyanins from Garcinia and Hibiscus species, reacting the hydroxycitric acid with IA and IIA group metals of periodic table, isolating the metal salt of hydroxycitric acid and drying and optionally mixing anthocyanins. Uses of the compositions that comprise extracts containing hydroxycitric acids (positive and negative isomers) and anthocyanins from Garcinia and Hibiscus species optionally along with secondary agents and pharmaceutically acceptable additives to manufacture a medicament for multiple therapeutic uses, as well as beverages and powdered beverage premixes, all of which provide healthful benefits, are also provided.

### RELATED APPLICATION

This application claims the benefit of and priority to U.S. Provisional Application Ser. No. 60/862,171, filed Oct. 19, 2006, the contents of which are incorporated by reference herein in its entirety.

### BACKGROUND

1. Field

The present invention relates to combination herbal compositions. More particularly, the present invention relates to herbal compositions that comprise extracts with controlled concentrations of hydroxycitric acid, (−) HCA and (+) HCA, from both Garcinia cambogia and Hibiscus sabdariffa, and anthocyanins from Hibiscus sabdariffa; processes for the preparation of these compositions, and methods for their therapeutic use.

### SUMMARY

Herbal compositions that comprise one or more of extracts of hydroxycitric acid, (−) HCA and (+) HCA, from Garcinia cambogia and Hibiscus sabdariffa, and anthocyanins from Hibiscus sabdariffa; all constituent extracts at independently controlled concentrations. These compositions may optionally include second therapeutic agents and pharmaceutically acceptable additives. The invention further provides processes for the preparation of these herbal compositions, such processes including steps of extracting hydroxycitric acids and anthocyanins from Garcinia and Hibiscus species, reacting the hydroxycitric acid with IA and IIA group metals of periodic table, isolating the metal salt of hydroxycitric acid and drying and optionally mixing anthocyanins to obtain the synergistic compositions. Uses of the compositions that comprise extracts containing hydroxycitric acids (positive and negative isomers) and anthocyanins from Garcinia and Hibiscus species optionally along with secondary agents and pharmaceutically acceptable additives to manufacture a medicament for multiple therapeutic uses, as well as beverages and powdered beverage premixes, all of which provide healthful benefits, are also provided.

An object of the present invention is to provide various metal salts of hydroxycitric acid (HCAs). Another object of the present invention is to provide metal salts using as a plant source Garcinia and Hibiscus species. Yet another object of the present invention is to provide a process for the preparation of various salts of HCAs from the extracts.

Another object of the present invention is to provide combination compositions of anthocyanins and HCAs from Garcinia and Hibiscus species, that may provide healthful or therapeutic effects. Still another object of the present invention is to provide compositions of the metal salts of hydroxycitric acid from Garcinia cambogia and Hibiscus sabdariffa both alone and in combination with further or second therapeutic and healthful agents, as exemplified by mangosteen, pomegranate, blueberries, grape seed, red bush, banaba, green tea, L-carnitine, chromium, and caffeine, to provide additive and synergistic therapeutic and healthful benefits. In the context of this patent application, “second agent” refers to agents that are derived from a source other than those of the “primary” agents (hydroxycitric acids and anthocyanins), which are derived from extracts of Garcinia and Hibiscus. Some of these further or second agents, such as mangosteen and grape seed, provide specific healthful benefits in the form of reducing the oxidative stress associated with reactive oxygen species, as may be measured by a test of oxygen radical absorbance capacity (ORAC). Some of these further agents may also include vitamins.

Another object of the present invention is to provide processes for preparing herbal compositions of the metal salts of hydroxycitric acids from Garcinia cambogia and Hibiscus sabdariffa, as well as processes for that combine these HCA compositions with healthful and therapeutic second agents. A related object of the present invention is to develop processes for the preparation of dry powder, tablets, and encapsulation, and in dry beverage mix, all from these various inventive compositions.

Another object of the present invention is to provide methods for the therapeutic use of the synergistic herbal compositions of the extracts of hydroxycitric acids from Garcinia and Hibiscus species, as well as anthocyanins from Hibiscus.

Still another object of the present invention is to provide compositions in solutions that are or controlled tonicity, including hypotonic, isotonic, and hypertonic solutions.

The above-mentioned features and objects of the present disclosure will become more apparent with reference to the following description.

### DETAILED DESCRIPTION

The present invention is in relation to combination herbal compositions for therapeutic use, wherein said compositions comprise of extracts containing hydroxycitric acids and anthocyanins from Garcinia and Hibiscus species, optionally along with healthful or therapeutics second agents, and pharmaceutically acceptable additives.

Hibiscus is also known as the “Roselle” flower a common name from German usage. Apart from the German “roselle”, the plant is known by numerous common names, including the French “karkadé” (red tea) the That “krachiap daeng”, the Persian “sour tea”, the Chinese “Lo Shen”, and the English “Roselle of Rama”, “Jamaica sorrel”, “Indian sorrel”, “jelly okra”, “sour-sour Queensland jelly plant”, and “Florida cranberry”.

These compositions in the form of various permutations and combinations of the extracts show additive or synergistic therapeutic activity with respect to the activities of the respective individual constituents. The details of preferred compositions as six therapeutic embodiments of the invention are provided in the table below.

The present disclosure provides, among other things, combination herbal compositions for therapeutic use, wherein said compositions comprise of extracts containing hydroxycitric acids and anthocyanins from Garcinia and Hibiscus species, optionally along with healthful or therapeutics second agents, and pharmaceutically acceptable additives.

These compositions in the form of various permutations and combinations of the extracts show additive or synergistic therapeutic activity with respect to the activities of the respective individual constituents. The details of preferred compositions as six therapeutic embodiments of the invention are provided in the table below.

| | Product Features |
|---|---|
| 1. | Hibiscus sabdariffa extract:- not less than 45% (+) HCA + 0.1 to |
| | 1.0% Anthocyanins, the anthocyanin content may include delphinidin |
| | 3-sambubioside. |
| 2. | (+) HCA Salts or free HCA |
| | a. various salts |
| | b. double or ternary or quaternary salts neutral or acidic (any of |
| | the IA group or group IIA metal salts) |
| | c. free HCA |
| 3. | Anthocyanins: 0.1% to 30% |
| 4. | (+) HCA salt + Anthocyanins from Product #3: the Ratio of HCA |
| | salt and Anthocyanins may be from 70:30 to 99:1 |
| 5. | Mix (−) HCA + (+) HCA Ratio 10%:90% to 90%:10% in salt form or |
| | liquid |
| 6. | Mix (−) HCA + (+) HCA and Anthocyanins from #3, the ratio of |
| | HCA salts or free HCA and Anthocyanins may be from 70:30 to 99:1 |

The present invention is in relation to combination herbal compositions for therapeutic use, wherein said compositions comprise of extracts containing hydroxycitric acids and anthocyanins from Garcinia and Hibiscus species, optionally along with healthful or therapeutics second agents, and pharmaceutically acceptable additives.

These compositions in the form of various permutations and combinations of the extracts show additive or synergistic therapeutic activity with respect to the activities of the respective individual constituents. The details of preferred compositions as six therapeutic embodiments of the invention are provided in the table below.

| | Product Features | |
|---|---|---|
| 1. | Hibiscus sabdariffa extract:- not less than 45% (+) HCA + 0.1 | |
| | to 1.0% Anthocyanins, the anthocyanin content may include | |
| | delphinidin 3-sambubioside. | |
| 2. | (+) HCA Salts or free HCA | |
| | a. | various salts |
| | b. | double or ternary or quaternary salts neutral or acidic (any of |
| | | the IA group or group IIA metal salts) |
| | c. | free HCA |

| | |
|---|---|
| 3. | Anthocyanins: 0.1% to 30% |
| 4. | (+) HCA Salt + Anthocyanins from Product #3: the Ratio of HCA |
| | salt and Anthocyanins may be from 70:30 to 99:1 |
| 5. | Mix (−) HCA + (+) HCA Ratio 10%:90% to 90%:10% in salt |
| | form or liquid |
| 6. | Mix (−) HCA + (+) HCA and Anthocyanins from #3, the ratio |
| | of HCA salts or free HCA and Anthocyanins may be from |
| | 70:30 to 99:1 |

In some embodiments of the present invention, the extract comprises of 45%-75% of Hydroxycitric acid and 0.1 to 30% anthocyanins.

Some embodiments include delphinidin 3-sambubioside and in the anthocyanin fraction. Delphinidin 3-sambubioside constitutes in the range of about 70% of the anthocyanins; cyanidin 3-sambubioside and other cyanidin and delphinidin glycosides may also be present. These two species account for a significant portion of the antioxidant activity obtained in the Hibiscus extracts.

In at least one embodiment of the present invention, the hydroxycitric acid is metal salt. In other embodiments of the present invention, the metals are selected from a group comprising IA and IIA group metals of the periodic table.

In at least one embodiment of the present invention, the ratio of hydroxycitric acid salts and anthocyanins is ranging from 70:30 and 99:1.

In yet other embodiments of the present invention, the ratios of the relative presence of (−) HCA, (+) HCA, and the anthocyanins are adjusted in order to optimize the inhibition of alpha amylase. In various embodiments, the concentrations of each of the constituents of embodiments of the composition are adjusted independently of each other, in order to provide particular or maximal therapeutic benefits.

In some embodiments of the present invention, the further or secondary agents are selected from a group comprising mangosteen, pomegranate, blueberries, grape seed, red bush, banaba, green tea, L-carnitine, chromium, caffeine, and vitamins.

In some embodiments of the present invention, the pharmaceutically acceptable additives are selected from a group comprising granulating agents, binding agents, lubricating agents, disintegrating agents, sweetening agents, coloring agents, flavoring agents, coating agents, plasticizers, preservatives, suspending agents, emulsifying agents and spheronization agents.

In some embodiments of the present invention, the therapeutic uses are obesity, hyperlipediemia, and any conditions for which suppression of appetite may provide healthful or therapeutic benefit. In a therapeutic manner, thus, patients or subjects suffering from these or related disorders are provided the therapeutic composition. In some embodiments, the compositions are administered as a beverage.

The present invention relates to processes for the preparation of combinational herbal compositions in at least some of the embodiments, such processes comprising steps of extracting hydroxycitric acids and anthocyanins from Garcinia and Hibiscus species, reacting the hydroxycitric acid with IA and IIA group metals of periodic table, isolating one or more salts of hydroxycitric acid, blending with anthocyanins from Hibiscus, and drying to obtain the therapeutic composition.

The present invention relates to use of compositions comprising extracts of hydroxycitric acid and anthocyanins from Garcinia and Hibiscus species optionally along with secondary agents and pharmaceutically acceptable additives to manufacture a medicament for multiple therapeutic uses.

The present invention further relates to providing therapeutic and dietary compositions in appropriate formulations, with excipients and coatings that support optimal and appropriate delivery and general bioavailability of the active agents of the compositions. Excipients may include granulating agents, binding agents, lubricating agents, disintegrating agents, sweetening agents, coloring agents, flavoring agents, coating agents, plasticizers, preservatives, suspending agents, emulsifying agents, and spheronization agents.

In some embodiments, nanotechnological approaches to controlling particle size, to the range of 20 microns, for example, and controlling surface features of the particles, all of which are directed toward increasing the bioavailability of such embodiments.

The technology of the present application is further elaborated with the following examples; these are provided for clarification of the invention, and should not be construed to limit the scope of the invention.

### EXAMPLES

### Example 1

### Process Description of Hibiscus Extract

1. Water or Hydro Alcohol extract of Hibiscus dried calyces, including three extractions with four volumes of solvent.

2. Combine all the three extracts (Extract #1), filter, concentrate, and dry to yield Product #1:

| (+) Hydroxycitric acid | Total Anthocyanins |
|---|---|
| ≧45% | 0.1%-1.0% by HPLC |

3. Load Extract #1 onto a weak anion column.

4. Wash the column with DM Water to remove the pigments.

5. Elute with sodium hydroxide solution.

6. Load the eluent onto a strong cation column.

7. Collect all the free acid (Extract #2).

8. Load Extract #1 onto a macroporous non ionic resin.

9. Collect the unadsorbed eluent, and wash the column with water.

10. Combine the eluent and washings (Extract #3).

11. Load the Extract #3 onto a weak anion column.

12. Wash the column with DM water to remove the pigments.

13. Elute with sodium hydroxide solution.

14. Load the eluent onto a strong cation column.

15. Collect all the free acid (Extract #4).

16. Bleach Extract #2 and/or Extract #4 with charcoal.

17. Filter the extract to remove charcoal (Extract #5).

18. React the Extract #5 with magnesium and potassium.

19. Concentrate and dry, to yield Product #2A:

| (+) HCA | Anthocyanins | Magnesium | Potassium | pH |
|---|---|---|---|---|
| ≧65% | <0.1% | ~4% | ~26% | 7-9 |

20. React the Extract #5 with calcium and potassium. Refer also to Note 1, below, regarding other salts of (+) HCA.

21. Concentrate and dry, to yield Product #2B:

| (+) HCA | Anthocyanins | Calcium | Potassium | pH |
|---|---|---|---|---|
| ≧65% | <0.1% | 8 ± 2% | 12-21% | 4-9 |

22. Concentrate the Extract #5 to 60% total solids, to yield Product #2C (liquid (+) HCA, not as a salt):

| (+) HCA | Anthocyanins | Ph |
|---|---|---|
| >65% | <0.1% | 1.8-2.5 |

23. Elute the Nonionic resin column with hydro alcohol.

24. Concentrate the eluent and dry to yield Product #3:

| (+)-Hydroxycitric acid | Total Anthocyanins |
|---|---|
| NMT 5% | 0.1%-30% by HPLC |

25. Combine Product #2A, 2B, or 2C with Product #4 (Product #4).

### Product #4A

### Salt+Anthocyanins (Product 3) any Salt Form

### Note

### Ratio of HCA Salt and Anthocyanins May Range Between 70:30 to 99:1

| (+) HCA | Anthocyanins | Magnesium | Potassium | pH |
|---|---|---|---|---|
| 45% to 65% | 0.1 to 10% | 2.8% to 4% | 18% to 26% | 4-7 |

### Product #4B

### Other (+) HCA Salts (Example Ca and K Salt)+Anthocyanins (Product 3)

| (+) HCA | Anthocyanins | Calcium | Potassium | pH |
|---|---|---|---|---|
| 45 to 75% | 0.1 to 10% | 5% to 12% | 10 to 17% | 4-7 |

### Product #4C

### (+) HCA Liquid+Anthocyanins (Product 3)

| (+) HCA | Anthocyanins | pH |
|---|---|---|
| 45%-65% | 0.1 to 10% | 1.5-2.5 |

### Example: 2 Process Description of Garcinia Extract

26. Water/Hydro Alcohol extract of Garcinia cambogia dried fruit rind. (Four extractions with six volumes of solvent.) Combine all the four extracts (Extract #6).

27. Load Extract #6 onto a weak anion column.

28. Wash the column with demineralized (DM) water to remove the pigments.

29. Elute with sodium hydroxide solution.

30. Load the eluent onto a strong cation column.

31. Collect all the free acid, and bleach the extract with charcoal.

32. Filter the extract to remove charcoal (Extract #7).

33. Combine Extract #5 and Extract #7 in the ratio 10%:90% to 90%:−10%, to yield Extract #8.

34. React the Extract #8 with magnesium and potassium.

35. Concentrate and dry (Product #5A) Mixture of (+) HCA and (−) HCA DDM Salt.

| (+) HCA & | | | | |
|---|---|---|---|---|
| (−) HCA | Anthocyanins | Magnesium | Potassium | pH |
| >65% | <0.1% | ~4% | ~26% | 7-9 |

36. React the Extract #8 with calcium and potassium.

37. Concentrate and dry (Product #5B). Note 1, other salts of a mixture of (+) HCA and (−) HCA.

| (+) HCA & | | | | |
|---|---|---|---|---|
| (−) HCA | Anthocyanins | Calcium | Potassium | pH |
| >65% | <0.1% | 8 + 2% | 12-21% | 4-6 |

38. Concentrate the Extract #8 to 60% total solids, to yield (Product #5C) liquid of a mixture of (+) HCA and (−) HCA, not as a salt.

| (+) HCA & (−) HCA | Anthocyanins | pH |
|---|---|---|
| >65% | <0.1% | 1.8-2.5 |

### Product #6A

### Salt+Anthocyanins (Product 3)

| (+) HCA & | | | | |
|---|---|---|---|---|
| (−) HCA | Anthocyanins | Magnesium | Potassium | pH |
| 45% to 65% | 0.1% to 10% | 2.8% to 4% | 18% to 26% | 4-7 |

### Product #6B

### CaK/Ca65+Anthocyanins

| (+) HCA & | | | | |
|---|---|---|---|---|
| (−) HCA | Anthocyanins | Calcium | Potassium | pH |
| 45 to 75% | 0.1 to 10% | 5% to 12% | 10% to 17% | 4-7 |

### Product #6C

### Garcitric Liquid+Anthocyanins Liquid of a Mixture of (+) HCA and (−) HCA, not as a Salt+Anthocyanins (Product 3)

| (+) HCA & (−) HCA | Anthocyanins | pH |
|---|---|---|
| 45%-65% | 0.1% to 10% | 1.5-2.5 |

In Example Two and other embodiments of the present invention, any of the IIA group metal, including but not limited to Be, Mg, Ca, Sr, Ba, or Ra; and any IA group metal including but not limited to Li, Na, K, Rb, Cs, or Fr can be used. The above data in the product #2A, 4A, 5A, 6A is for a specific Mg/K salt. Any of the above metals can be used to give salts as any double metal salt, tri metal salt or even tertiary metal salt as monomers, dimers, trimers, etc., and esters of (−) HCA and/or (+) HCA, as applicable.

While the apparatus and method have been described in terms of what are presently considered to be the most practical and preferred embodiments, it is to be understood that the disclosure need not to be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A combination herbal composition for therapeutic use, the composition comprising: extracts of hydroxycitric acid and anthocyanins from Garcinia and Hibiscus species.

2. The composition of claim 1, wherein hydroxycitric acid comprises both positive and negative isomers, each isomer at an independently controlled concentration.

3. The composition of claim 1, wherein the hydroxycitric acid and the anthocyanins are at concentrations controlled independently of each other.

4. The composition of claim 1, further comprising one or more second agents and pharmaceutically acceptable additives.

5. The composition of claim 1, further comprising pharmaceutically acceptable additives.

6. The composition of claim 1, wherein the composition is in a liquid formulation, the liquid having a tonicity being any of hypotonic, isotonic, or hypertonic.

7. The composition of claim 1, wherein the composition comprises about 45% to about 75% of hydroxycitric acid and about 0.1% to about 30% anthocyanins.

8. The composition of claim 1, wherein the hydroxycitric acid is a metal salt.

9. The composition of claim 1, wherein the metals are selected from a group comprising IA and IIA group metals of the periodic table.

10. The composition of claim 1, wherein the percentage ratio of hydroxycitric acid salts to anthocyanins ranges from about 70:30 to about 99:1.

11. The composition of claim 1, wherein the anthocyanins include delphinidin 3-sambubioside.

12. The composition of claim 2, wherein the one or more second agents are selected from a group consisting of mangosteen, pomegranate, blueberries, grape seed, red bush, banaba, green tea, L-carnitine, chromium, vitamins, and caffeine.

13. The composition of claim 3, wherein the pharmaceutically acceptable additives are selected from a group consisting of granulating agents, binding agents, lubricating agents, disintegrating agents, sweetening agents, coloring agents, flavoring agents, coating agents, plasticizers, preservatives, suspending agents, emulsifying agents, and spheronization agents.

14. The composition of claim 1, wherein the therapeutic uses are for the treatment of a metabolic disorder, the disorder including any of obesity, hyperlipidemic, or any condition for which suppressing appetite provides therapeutic benefit.

15. A process for the preparation of a combination herbal composition comprising:
a) extracting hydroxycitric acid (−) HCA from Garcinia cambogia, hydroxycitric acid (+) HCA from Hibiscus sabdariffa, and anthocyanins from Hibiscus;
b) reacting one or more hydroxycitric acids with or without anthocyanins, with IA and IIA group metals of periodic table;
isolating the metal salts of hydroxycitric acids; and
drying to obtain the herbal compositions.

16. A method for the manufacture of a combination herbal composition comprising the combining of extracts of hydroxycitric acid (−) HCA from Garcinia cambogia, hydroxycitric acid (+) HCA from Hibiscus sabdariffa, and anthocyanins from Hibiscus.

17. The method of claim 16, further comprising combining the composition with second agents, the second agents being any combination of mangosteen, pomegranate, blueberries, grape seed, red bush, banaba, green tea, L-carnitine, chromium, or caffeine.

18. A method of treating a subject suffering from a metabolic disorder, the method comprising administering the subject a composition that includes extracts of hydroxycitric acid (−) HCA from Garcinia cambogia, hydroxycitric acid (+) HCA from Hibiscus sabdariffa, and anthocyanins from Hibiscus.
